(19) United States
(12) Patent Application Publication
(10) Pub. No.: US 2025/0090734 A1
(43) Pub. Date: Mar. 20, 2025

(54) OPEN-CLOSED LOOP PERFUSION RESERVOIR

(71) Applicant: MUSC Foundation for Research Development, Charleston, SC (US)

(72) Inventors: David Clyde Fisher, JR., Mt. Pleasant, SC (US); David Christopher Fitzgerald, Mt. Pleasant, SC (US)

(21) Appl. No.: 18/807,379

(22) Filed: Aug. 16, 2024

Related U.S. Application Data
(60) Provisional application No. 63/578,229, filed on Aug. 23, 2023.

(51) Int. Cl. A61M 1/14; A61M 1/16; A61M 1/36; B01D 35/147

(52) U.S. Cl. CPC A61M 1/1563; A61M 1/1565; B01D 35/147; A61M 1/1698; A61M 1/3666; B01D 2201/202; B01D 2201/204

(57) ABSTRACT

Aspects of the present invention relate to a blood perfusion reservoir device including a housing forming proximal and distal chambers, the housing having proximal and distal openings, a valve having a proximal end, a distal end and a length forming a lumen therebetween, the valve being positioned within the housing such that the proximal end passes through the proximal opening of the housing, the distal end passes through the distal opening of the housing, and the length passes through the proximal and distal chambers of the housing, wherein the valve is configurable between a first and second position, the first position forming a first flow path and the second position forming a second flow path, a filter reservoir having a frame with a filter media forming proximal and distal filter regions with a proximal opening, and positioned within the housing fixedly attached to the valve and surrounding a length of the valve, wherein the first flow path fluidly connects the proximal and distal openings, and the flow bypasses the proximal and distal chambers, the proximal and distal filter regions, and the second flow path fluidly connects the proximal and distal openings, and the flow passes through proximal and distal chambers and the proximal and distal filter regions.

### CROSS-REFERENCE TO RELATED APPLICATIONS

This application claims priority to U.S. Provisional Application No. 63/578,229 filed on Aug. 23, 2023, incorporated herein by reference in its entirety.

### BACKGROUND OF THE INVENTION

During cardiopulmonary bypass, a system is configured to filter, oxygenate, and pump blood to the patient while their heart and lungs are being bypassed. There are two types of systems: a closed-loop bypass and an open-loop bypass. A closed loop system lowers the heparin requirement and reduces the blood/foreign surface interface but doesn't allow for controlling of patient blood volume. An open loop system allows for control of patient blood volume (in some cases, via vacuum applied to reservoir) but increases the heparin requirement and the blood/foreign surface interface. Each system has its purpose, but currently, they only exist as systems that are meant to be used independently from each other. There are cases when a surgeon wants the ability to convert between an open and closed loop system. Attempts to reconfigure existing products, unfortunately, require additional tubing, cuts to the sterile blood pathway, and several tubing clamps, all of which introduce a risk of infection and air entrainment posing a serious threat to the patient.

Thus, there is a need in the art for a combined closed-open loop bypass system that may filter, oxygenate, and pump blood to a patient during cardiopulmonary bypass. The present invention satisfies that need.

### SUMMARY OF THE INVENTION

Aspects of the present invention relate to a blood perfusion reservoir device including a housing forming proximal and distal chambers, the housing having proximal and distal openings, a valve having a proximal end, a distal end and a length forming a lumen therebetween, the valve being positioned within the housing such that the proximal end passes through the proximal opening of the housing, the distal end passes through the distal opening of the housing, and the length passes through the proximal and distal chambers of the housing, wherein the valve is configurable between a first and second position, the first position forming a first flow path and the second position forming a second flow path, a filter reservoir having a frame with a filter media forming proximal and distal filter regions with a proximal opening, and positioned within the housing fixedly attached to the valve and surrounding a length of the valve, wherein the first flow path fluidly connects the proximal and distal openings, and the flow bypasses the proximal and distal chambers, the proximal and distal filter regions, and the second flow path fluidly connects the proximal and distal openings, and the flow passes through proximal and distal chambers and the proximal and distal filter regions.

In some embodiments, the valve comprises at least one outer sheath and at least one inner sheath, with the inner sheath inserted into the outer sheath and slidably movable relative to the outer sheath and forming the lumen therethrough, each sheath comprising one or more distal openings, and one or more lateral openings along the length of each sheath.

In some embodiments, the sheaths when configured to the first position of the valve allow the first flow path enabling fluid communication between the proximal and distal openings of the housing through the one or more distal openings of the sheaths.

In some embodiments, the sheaths when configured to the second position of the valve allow the second flow path enabling fluid communication between the proximal and distal openings of the housing through the one or more lateral openings of the sheaths.

In some embodiments, the first flow path is defined as a fluidly connected path entering the proximal opening of the housing, proceeding through the lumen formed by the inner sheath, exiting the distal opening of the inner sheath and entering the outer sheath, proceeding through the lumen formed by the outer sheath and exiting the distal opening of the outer sheath, and out the distal opening of the housing.

In some embodiments, the second flow path is defined as a fluidly connected path entering the proximal opening of the housing, proceeding through the lumen formed by the inner sheath, exiting the lumen of the inner sheath through the one or more lateral openings, passing through the one or more lateral openings of the outer sheath into the distal filter region and filling the proximal filter region as necessary, through the filter media, into the distal chamber and filling the proximal chamber as necessary, and out the distal opening of the housing.

In some embodiments, the outer sheath slidably moves axially between the first and second positions of the valve.

In some embodiments, the housing and filter reservoir comprise proximal openings configured to receive one or more lids covering the proximal openings.

In some embodiments, the proximal openings of the inner and outer sheaths extend proximally through the one or more lids.

In some embodiments, the device further comprises at least one opening in the housing configured to apply pressure or vacuum to the first and second interior volumes.

In some embodiments, the device further comprises a proximal button fixedly attached to a position at the proximal end of the outer sheath, configured to lift or depress the outer sheath to configure the valve between the first and second positions, respectively. In some embodiments, the device further comprises a spring positioned between the button and the housing, configured to apply a preload to the button.

In some embodiments, the housing is a shape selected from the group consisting of: frustoconical, cone, cylinder, sphere, and hexahedron.

In some embodiments, the filter reservoir is a shape selected from the group consisting of: frustoconical, cone, cylinder, sphere, and hexahedron.

In some embodiments, the housing comprises a material selected from the group consisting of: plastic, metal, composite, glass, ABS, PLA, TPU, PP, PVC, polycarbonate, acrylic, nylon, polyethylene, polytetrafluoroethylene (Teflon), and polyethylene terephthalate (Dacron).

In some embodiments, the filter reservoir comprises a material selected from the group consisting of: plastic, metal, composite, glass, ABS, PLA, TPU, PP, PVC, polycarbonate, acrylic, nylon, polyethylene, polytetrafluoroethylene (Teflon), and polyethylene terephthalate (Dacron).

In some embodiments, the housing has a proximal to distal length ranging between 20 and 50 cm, and a width or diameter ranging between 10 and 40 cm.

In some embodiments, the filter reservoir has a proximal to distal length ranging between 10 and 40 cm, and a width or diameter ranging between 5 and 30 cm.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIGS. 1A through 1C depict an exemplary open-closed loop perfusion reservoir device configured for a first flow path according to aspects of the present invention. FIG. 1A depicts a side view of the device. FIG. 1B depicts a side cut-away view of the device. FIG. 1C depicts an enlarged side view of the distal end of the device.

FIGS. 2A through 2C depict an exemplary open-closed loop perfusion reservoir device configured for a second flow path according to aspects of the present invention. FIG. 2A depicts a side view of the device. FIG. 2B depicts a side cut-away view of the device. FIG. 2C depicts an enlarged side view of the distal end of the device.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The terms “proximal,” “distal,” “anterior,” “posterior,” “medial,” “lateral,” “superior,” and “inferior” are defined by their standard usage indicating a directional term of reference. For example, “proximal” refers to an upper location from a point of reference, while “distal” refers to a lower location from a point of reference. In another example, “anterior” refers to the front of a body or structure, while “posterior” refers to the rear of a body or structure. In another example, “medial” refers to the direction towards the midline of a body or structure, and “lateral” refers to the direction away from the midline of a body or structure. In some examples, “lateral” or “laterally” may refer to any sideways direction. In another example, “superior” refers to the top of a body or structure, while “inferior” refers to the bottom of a body or structure. It should be understood, however, that the directional term of reference may be interpreted within the context of a specific body or structure, such that a directional term referring to a location in the context of the reference body or structure may remain consistent as the orientation of the body or structure changes.

The terms “patient,” “subject,” “individual,” and the like are used interchangeably herein, and refer to any animal amenable to the systems, devices, and methods described herein. The patient, subject or individual may be a mammal, and in some instances, a human.

The terms “fluid” or “liquid medium” are used herein to refer to a substance which is in the form of a liquid at ambient temperature or room temperature. Non-limiting examples of fluid include: water, blood, blood serum, body fluids, oils, milk, and any combination thereof.

The term “membrane” is used herein to refer to a film capable of performing separations. The separation may be absolute (i.e., non-permeable membrane), selective (i.e., semi-permeable membrane), or limited (i.e., permeable membrane).

The terms “filter” or “filter medium” are used herein to refer to any medium suitable for physical separation of liquids and solids. Filter medium may include substrates having pores sized to exclude passage of solid particles, while allowing passage of smaller liquid molecules (e.g., filter cloths, membranes and the like). Filter medium also includes substrates comprising a plurality of particles, such that the particles serve as a physical barrier to the passage of other solid particles (e.g., diatomaceous earth and the like). Non-limiting examples of filter media include: PES (polyethersulfone) membranes, cellulose, cellulose acetate and regenerated cellulose membranes (i.e., typical paper filters), polypropylene membranes/cloth, Teflon and other fluoropolymer (hydrophilic and hydrophobic) membranes, glass fibers or fritted glass, other polymer membranes (e.g., polyester), metal mesh, charcoal, powdered activated carbon (PAC), graphite, graphene, graphene oxide, manganese oxides (MnOx), manganese sulfides (MnSx), molybdenum oxides (MoOx), molybdenum sulfides (MoSx), silicon oxides (SiOx), silicon sulfides (SiSx), aluminum oxides (AlyOz), aluminum sulfides (AlySz), boron oxides (ByOz), zeolites, tungsten diselenide (WSe2), niobium disclenide (NbSc2), boron nitride (BN), tungsten sulfide (WS2), phosphorene (PR3), tin (Sn), and transition metal di-chalcogenides.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

### Open-Closed Loop Blood Perfusion Reservoir Device

Aspects of the present invention relate to an open-closed loop blood perfusion reservoir device for combining a closed loop and open loop bypass system for use, in one example, during a cardiopulmonary bypass. An open loop bypass system allows for control of patient blood volume (in some cases, via vacuum applied to reservoir) but increases the heparin requirement and the blood/foreign surface interface. A closed-loop bypass system lowers the heparin requirement and reduces the blood/foreign surface interface but does not allow for control of patient blood volume. When combined, advances represented by the embodiments described herein provide a hybrid device where a single venous reservoir includes both open and closed-loop modalities. Embodiments of the devices described herein are uniquely configured and suitable for blood perfusion protocols during procedures such as cardiothoracic surgery.

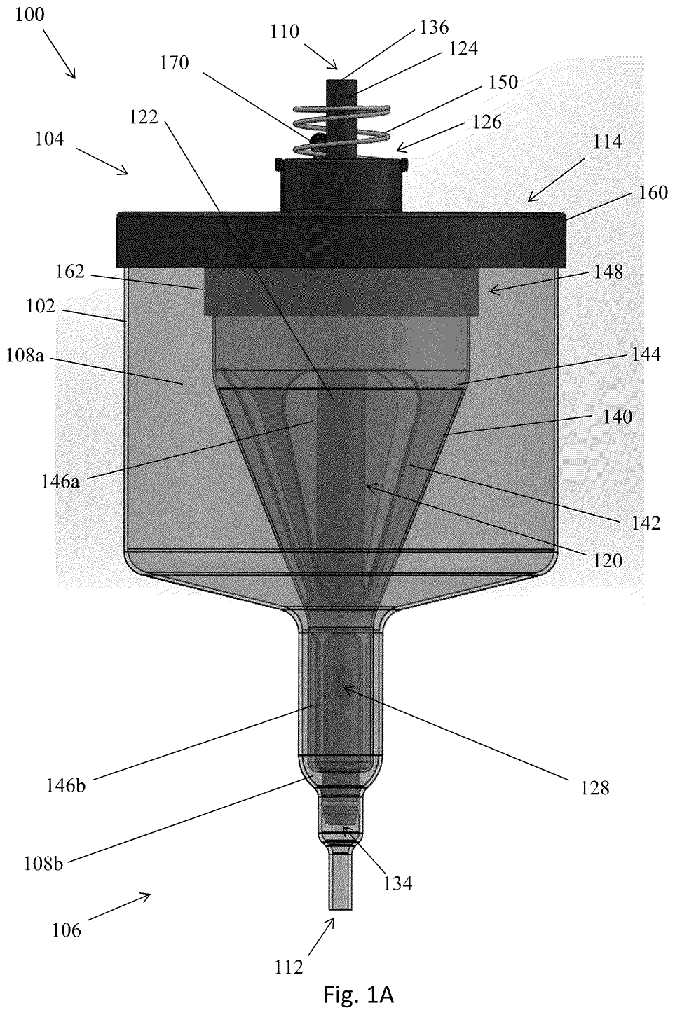

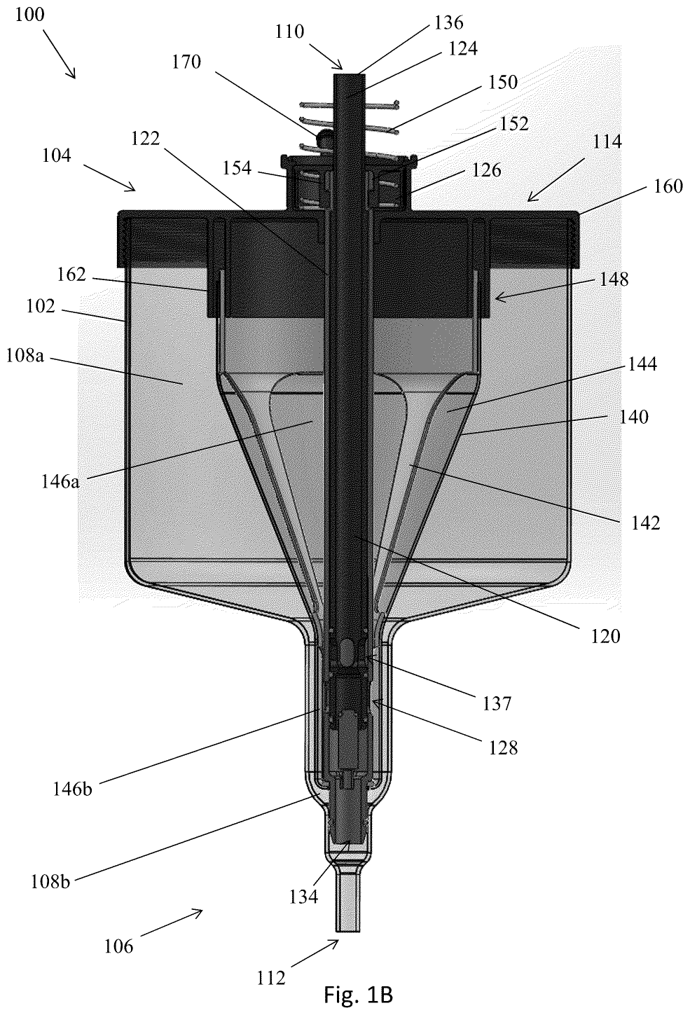

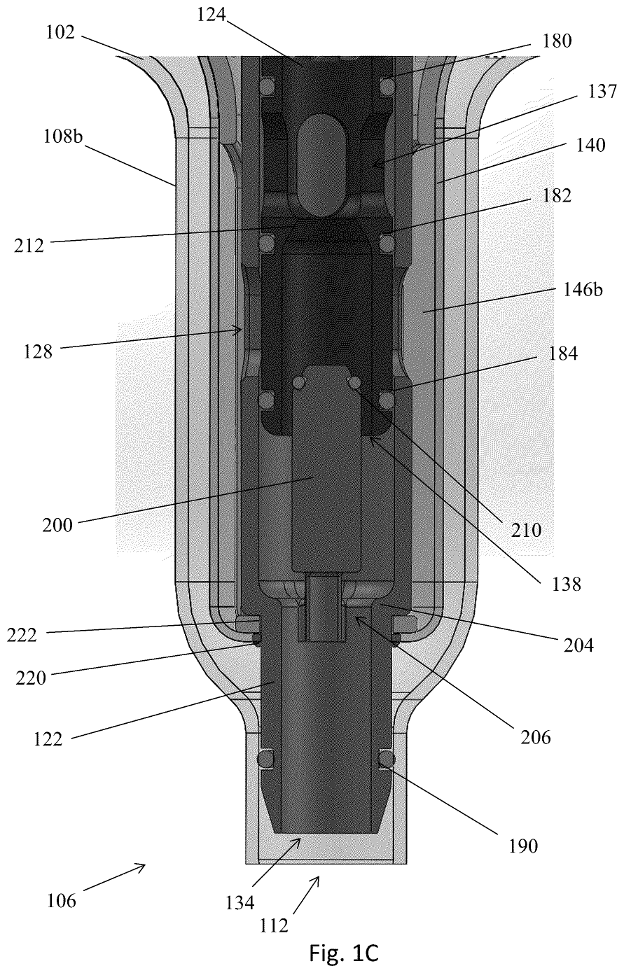

Aspects of the present invention relate to an open-closed loop blood perfusion reservoir device 100 as discussed herein. Referring now to FIG. 1A, shown is an exemplary open-closed loop blood perfusion reservoir device 100 comprising a housing 102 having a proximal end 104 and a distal end 106 forming a proximal chamber 108a and a distal chamber 108b within. Device 100 is controlled and/or actuated by adjusting a valve 120 (e.g., a slide valve) that extends from proximal end 104 to distal end 106 within the housing. By adjusting valve 120, at least two flow paths may be established through device 100. In general, device 100 comprises at least one proximal opening or inlet 110 and at least one distal opening or outlet 112, wherein portions of valve 120 are positioned near to, and fluidly connecting with each opening. Device 100 further comprises a filter reservoir 140 positioned within housing 102 connected to, and at least partially surrounding valve 120. By adjusting or positioning valve 120, the flow paths may be established that either pass through, or by-pass the filter reservoir 140. In some embodiments, device 100 comprises a removable proximal lid 160 scaling proximal end 104 of housing 102. In some embodiments, the proximal lid 160 comprises an adjustment mechanism for adjusting or positioning valve 120. In some embodiments, device 100 further comprises an opening 170 configured to apply vacuum or pressure to housing 102. With these features, device 100 may be adjusted to establish flow paths defining open loop and closed loop modalities for blood perfusion by adjusting valve 120 and/or applying vacuum or pressure to the device via opening 170.

In some embodiments, the disclosed device provides a single venous reservoir (ie., housing 102) that can seamlessly switch between open and closed loop configurations without the need for reconfiguration of a perfusion circuit and/or tubing. The unique design of housing 102, valve 120 and filter reservoir 140 allows the switching between open and closed loop modalities without causing pooling, stagnation of flow, or entrainment of air.

Aspects of the present invention relate to valve 120 positioned within housing 102 and providing one or more flow paths between the openings of the device. In some embodiments, valve 120 extends through at least a portion of housing 102 (e.g., positioned at least partially within proximal chamber 108a and/or distal chamber 108b) and fluidly connects to one or more openings in device 100. In some embodiments, valve 120 comprises at least one outer sheath 122 and at least one slidable inner sheath 124 that is movable relative to outer sheath 122, and positionable in at least two configurations or positions (i.e., open and closed, or first and second positions). In some embodiments, outer sheath 122 and inner sheath 124 each comprise proximal and distal ends, and lengths therebetween forming lumens extending within, with one or more openings (lateral or axial) in the sheaths that may be opened or occluded to provide one or more flow paths from inlet 110 to outlet 112. When inner sheath 124 is slidably inserted into outer sheath 122, a single lumen is formed therethrough (i.e., a flow path is established through the lumen of inner sheath 124).

The unique design of housing 102 uses gravity and a tapered shape to pool or concentrate blood near outlet 112 and prevent turbulence within the housing when switching modalities (e.g., between open loop and a closed loop configurations). In some embodiments, distal chamber 108b comprises a proximal end, a distal end, and a length therebetween forming a volume or tubular structure for distal end 106 of housing 102. In some embodiments, proximal chamber 108a is formed separate from, but fluidly connected to distal chamber 108b. In some embodiments, proximal chamber 108a may be configured to hold a larger volume than distal chamber 108b. In some embodiments, proximal chamber 108a spills or directs flow into distal chamber 108b.

Aspects of the present invention relate to a filter reservoir 140 designed to filter fluid passing through device 100 when a flow path is established through the filter. In some embodiments, filter reservoir 140 is disposed and/or positioned across proximal chamber 108a and distal chamber 108b, and comprises a filter frame 142, a filter mesh or media 144 forming a proximal filter region 146a and distal filter region 146b. In some embodiments, proximal filter region 146a is formed separate from but fluidly connected to distal filter region 146b. In some embodiments, filter reservoir 140 surrounds and/or encloses at least a portion of the length of valve 120. In some embodiments, filter reservoir 140 extends outwardly from, and is fixedly, removably and/or sealingly attached to a position along the length of outer sheath 122 of valve 120. In some embodiments, filter reservoir 140 is designed and shaped to fit within and align with the design and shape of housing 102.

Aspects of the present invention relate to a removable lid for closing and/or scaling at least a portion of housing 102 (e.g., an opening at proximal end 104 of housing 102). In some embodiments, housing 102 comprises a proximal opening 114 at proximal end 104. In some embodiments, device 100 comprises a proximal lid 160 having a proximal top surface and distal bottom surface. In some embodiments, proximal lid 160 is configured to fixedly and removably attach to, and sealingly close, at least a portion of proximal opening 114. Similar to housing 102, filter reservoir 140 comprises a proximal opening 148 that can interface with and be sealed and/or closed by proximal lid 160. For example, in some embodiments, proximal lid 160 is configured to fixedly and removably attach to, and scalingly close, at least a portion of proximal opening 148 of filter reservoir 140. In some embodiments, proximal lid 160 may fixedly, releasably and scalingly attach to housing 102 and/or filter reservoir 140 by any method or mechanism known to one of ordinary level of skill in the art. For example, but without limitation, proximal lid 160 may attach with a threaded portion, a compression fit, a friction fit, a tongue-and-groove mechanism, an indent and detent mechanism, a locking mechanism, or the like, and any combinations thereof. In some embodiments, either housing 102, filter reservoir 140 and/or proximal lid 160 may comprise one or more o-rings and/or gaskets positioned between the interfaces thereof in order to form an air- and liquid-tight hermetic seal. In some embodiments, proximal lid 160 comprises one or more retaining portions 162 extending in a distal direction from the bottom surface of the lid for retaining at least a portion of filter reservoir 140. In some embodiments, the proximal end of filter reservoir 140 is retained by retaining portion 162 of proximal lid 160, such that filter reservoir 140 is axially aligned within housing 102. In some embodiments, when valve 120 is configured between its first and second positions, filter reservoir 140 translates axially from a first proximal position to a second distal position within housing 102.

Aspects of the present invention relate to the design and configuration of valve 120 within housing 102. In some embodiments, valve 120 comprises concentric sheaths (e.g. outer sheath 122 and inner sheath 124) wherein inner sheath 124 is slidably and releasably inserted into outer sheath 122. In some embodiment, distal portions of valve 120 are slidably attached to interior positions of housing 102 via friction or compression fit, and one or more o-rings positioned to interface valve 120 to interior portions of housing 102. In some embodiments, proximal portions of valve 120 are connected or attached to portions of device 100 near proximal end 104.

In some embodiments, inner sheath 124 is inserted coaxially into outer sheath 122, wherein outer sheath 122 is configured to slide or axially translate along at least a portion of the length of inner sheath 124. In some embodiments, at least a portion of valve 120, outer sheath 122 and/or inner sheath 124 may pass through proximal lid 160. In some embodiments, the proximal end of outer sheath 122 encloses at least a portion of the proximal end of inner sheath 124, and a protruding section of inner sheath 124 forms at least a portion of inlet 110. In some embodiments, when outer sheath 122 is axially translated along the length of inner sheath 124, valve 120 is configured between at least first and second positions, creating at least first and second flow paths for fluids from inlet 110 to outlet 112 as discussed herein.

Generally, valve 120 may be configured to at least first and second positions by translating outer sheath 122 axially along the length of inner sheath 124. In some embodiments, valve 120 is configured, actuated and/or translated axially using an actuator 126 comprising a peripheral flange or lever extending outward radially from the sides of outer sheath 122 proximal to lid 160. In some embodiments, a user may push down or lift up on actuator 126 to configure valve 120 between first and second positions, respectively.

Referring now to FIGS. 1A through 1C, shown is an exemplary device 100 with outer sheath 122 and inner sheath 124 forming portions of valve 120. In some embodiments, outer sheath 122 and inner sheath 124 comprise one or more openings (e.g., axial and/or lateral openings) that enable at least first and second flow paths through valve 120 and housing 102 when the slide valve is adjusted. In some embodiments, outer sheath 122 comprises a proximal axial opening at the proximal end of the sheath, one or more lateral openings 128 along the length of sheath, and a distal axial opening 134 at the distal end of the sheath. In some embodiments, the one or more lateral openings 128 are positioned within or aligned with distal filter region 146b. In some embodiments, outer sheath 122 terminates with distal axial opening 134 positioned near and in fluid communication with outlet 112 of housing 102.

Referring now in detail to FIGS. 1B & 1C, a side cutaway view of device 100 is shown. In some embodiments, inner sheath 124 comprises a proximal opening 136 that forms at least a portion of inlet 110 for device 100. In some embodiments, inner sheath 124 comprises one or more lateral opening 137 along the length of the sheath, distal to opening 136. In some embodiments, inner sheath 124 further comprises one or more distal axial openings 138 in the distal end of the sheath, distal to lateral openings 137. In some embodiments, outer sheath 122 and inner sheath 124 comprise one or more lateral circumferential recesses in the exterior surfaces or outer walls of the sheaths configured to receive and/or retain one or more o-rings or gaskets. Referring now to FIG. 1C in detail, in some embodiments inner sheath 124 comprises a first lateral recess 180, configured to receive a first o-ring, a second lateral recess 182, distal to recess 180, configured to receive a second o-ring, and a third lateral recess 184, distal to recess 182, configured to receive a third o-ring.

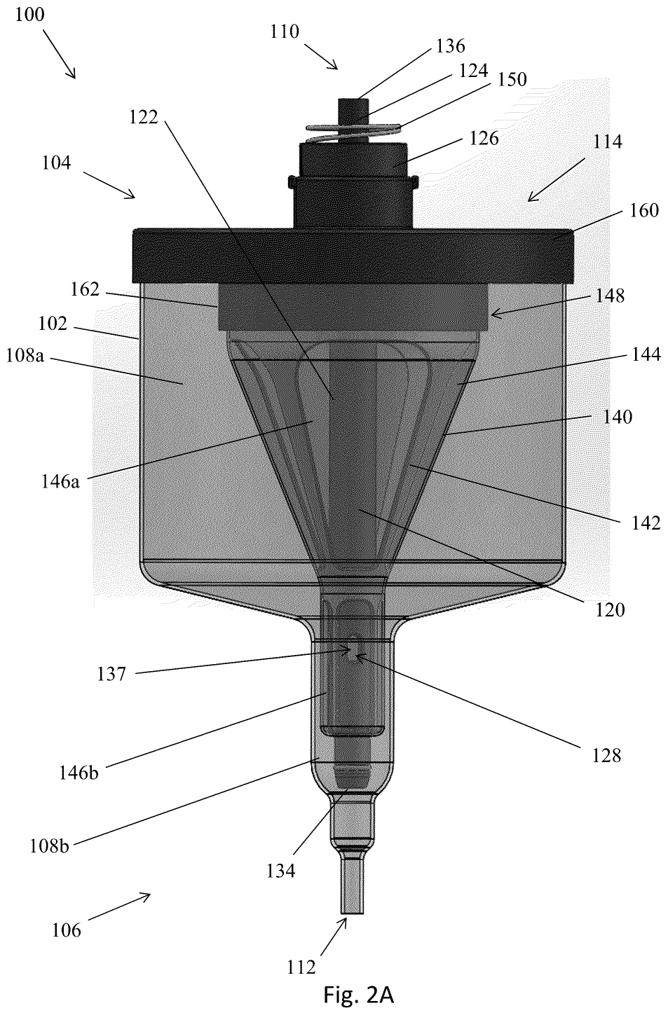

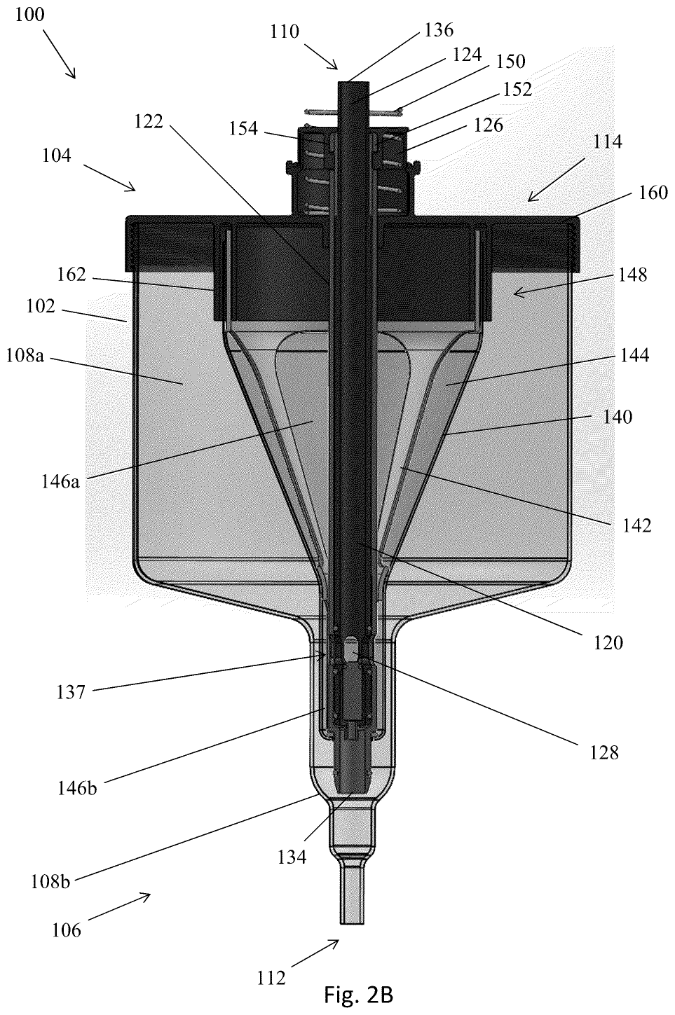

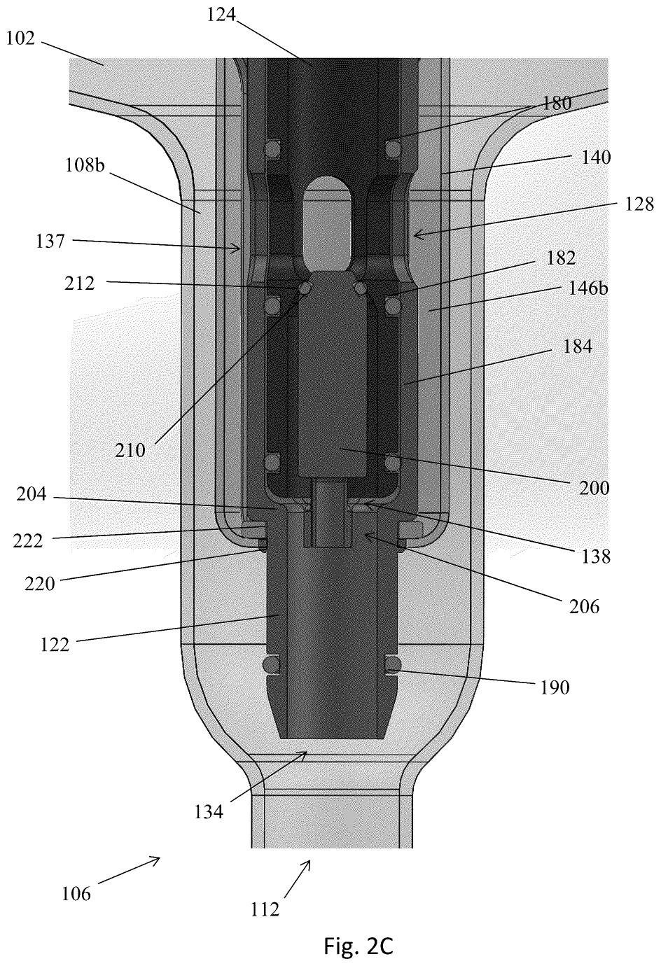

In some embodiments, outer sheath 122 comprises one or more internal features or structures for allowing or restricting flow through valve 120. Referring again to FIG. 1C and now also FIG. 2C, in some embodiments, outer sheath 122 comprises a post 200 connected to an internal flange 204. In some embodiments, post 200 comprises a proximal end attached to flange 204 and a distal end extending in a proximal direction into the lumen of outer sheath 122. It should be appreciated that when slidable valve 120 is assembled with outer sheath 122 and inner sheath 124, a portion of post 200 extends into opening 138 of inner sheath 124.

Post 200 may be used to occlude a flow path of valve 120. In some embodiments, flange 204 forms an internal opening 206 surrounding post 200 and fluidly connecting the proximal and distal ends of outer sheath 122. In some embodiments, axial opening 138 of inner sheath 124 further comprises a neck 212 configured to interface with, and create a seal with a tapered or chamfered portion of post 200. In some embodiments, post 200 comprises an o-ring positioned in a lateral circumferential recess or indent 210 on the portion of post 200 configured to interface with neck 212. In some embodiments, inner sheath 124 comprises at least a fourth lateral recess 190 in a distal portion of the sheath configured to receive a fourth o-ring. In some embodiments, the fourth lateral recess 190 and the fourth o-ring are positioned to sealingly contact an inside surface of housing 102 distal to distal chamber 108b and proximal to outlet 112. It should be appreciated that any o-ring and/or gasket material known in the art may be used with device 100 (e.g., in the aforementioned lateral recesses (i.e. lateral recesses 180, 182, 184, and 190)).

Referring now again to FIGS. 1A through IC, shown is an exemplary device 100 wherein valve 120 is configured to a first position, and forms a first flow path connecting inlet 110 and outlet 112. In some embodiments, when valve 120 is configured to a first position (in some examples by depressing down actuator 126), outer sheath 122 and inner sheath 124 form a first flow path that fluidly connects inlet 110 and outlet 112, wherein the flow bypasses filter reservoir 140, proximal chamber 108a, distal chamber 108b, filter region 146a, and distal filter region 146b. When valve 120 is configured to the first position, fluid enters inlet 110 and opening 136 and travels down through the lumen of inner sheath 124, bypasses one or more lateral opening 137 (as lateral opening 137 is occluded by the inner wall of outer sheath 122, when valve 120 is configured in the first position), bypasses one or more lateral opening 128 (as lateral opening 128 is occluded by the distal end sidewalls of inner sheath 124, and the second and third o-rings of inner sheath 124), passes through distal opening 138 and enters the lumen formed by the distal end of outer sheath 122, and proceeds to exit outer sheath 122 and finally out the outlet 112 of housing 102.

Referring now to FIGS. 2A through 2C, shown is an exemplary device 100 wherein valve 120 is configured to a second position, and forms a second flow path connecting inlet 110 and outlet 112. In some embodiments when valve 120 is configured to a second position (in some examples by depressing or lifting up actuator 126), valve 120 forms a second flow path that fluidly connects inlet 110 and outlet 112 wherein the flow passes through the interior chambers of housing 102 and regions of filter reservoir 140 (i.e., proximal filter region 146a, distal filter region 146b, proximal chamber 108a, and distal chamber 108b). When valve 120 is configured to the second position, fluid enters inlet 110 and opening 136 and travels distally through the lumen of inner sheath 124, proceeds out of inner sheath 124 (and outer sheath 122) through lateral openings 137 and 128 (as lateral openings 137 and 128 are aligned and fluidly connected when valve 120 is configured to the second position). It should be appreciated that when valve 120 is in the second position, neck 212 and the o-ring on post 200 occlude opening 138. Upon exiting the valve 120 through the lateral openings, the fluid enters distal filter region 146b of filter reservoir 140, files proximal filter region 146a as necessary, and passes through filter mesh 144 of filter reservoir 140. The fluid then enters or spills over into distal chamber 108b of housing chamber 102, fills proximal chamber 108a as necessary, and proceeds distally in housing chamber 102 finally exiting out through outlet 112.

Aspects of the present invention relate to a filter positioned and/or disposed within housing 102 of device 100. Referring again to FIG. 1C and FIG. 2C, valve 120 and/or outer sheath 122 may comprise features for affixing and/or sealing filter reservoir 140 in order to partition proximal chamber 108a and distal chamber 108b from proximal filter region 146a and distal filter region 146b. In some embodiments, outer sheath 122 comprises a flange 220 extending outward radially from the outer surface of the sheath, distal to lateral openings 128 and forming a recess 222 wherein a distal portion of filter reservoir 140 may fixedly, rotatably, scalingly and/or removably attach to valve 120 and/or outer sheath 122. It should be understood that the interface between flange 220, recess 222 and filter reservoir 140 may comprise o-rings, gaskets, glues, and the like, in order to provide a hermetic seal between filter reservoir 140 and valve 120.

Referring now to FIG. 1B, in some embodiments, actuator 126 is a push button configured to retain a spring 150 and at least a portion of outer sheath 122, wherein depressing or pushing down the button positions actuator 126 in a first position, configuring valve 120 to its first position, and forming the first flow path. Depressing actuator 126 again releases the spring-loaded button from the first position, and positions the actuator 126 in a second position, configuring valve 120 to its second position and forming the second flow path. In some embodiments, outer sheath 122 and actuator 126 comprise interlocking or interfacing features for fixedly attaching the lever to the sheath. In some embodiments, outer sheath 122 comprises a proximal flange 152 extending outward from the outside surface of the sheath, configured to at least partially extend into and be retained by a recess 154 in actuator 126. In some embodiments, valve 120 comprises a distal lateral recess 190 in outer sheath 122 comprising an o-ring and configured to sealingly contact the distal inside surface of housing 102, distal to distal chamber 108b, and proximal to outlet 112.

Aspects of the present invention relate to applying pressure or vacuum to device 100 in order to modulate the fluid flow through the device, change the amount of fluid held within the housing of the device, and/or limit or increase the amount of fluid entering the subject. In some embodiments, device 100 further comprises an opening 170 passing through lid 160 configured to apply pressure or vacuum to the interior volume of housing 102. It should be appreciated that any vacuum or pressure pump known in the art may be used to modulate fluid flow through or within device 100.

Aspects of the present invention relate to the housing and/or filter of device 100 being at least partially formed in one or more shapes and having specific dimensions to enable blood perfusion of a subject. For example, but without limitation, housing 102 and/or filter reservoir 140 may be at least partially formed in any shape or combinations of shapes known in the art. For example, in some embodiments, housing 102 and/or filter reservoir 140 comprise frustoconical shapes, round shapes, rectangular shapes, hexagonal shapes, circular shapes, spherical shapes, and any combinations thereof.

In some embodiments, housing 102 has a length, measured from proximal end 104 to distal end 106, ranging between 10 cm and 100 cm, 20 cm and 80 cm, 30 cm and 60 cm, and 40 cm and 50 cm. For example, in some embodiments, housing 102 has a length of about 45 cm. In some embodiments, housing 102 has a width or diameter ranging between 10 cm and 60 cm, 20 cm and 50 cm, and 30 cm and 40 cm. For example, in some embodiments housing 102 has a width of about 35 cm.

In some embodiments, filter reservoir 140 has a length, measured from proximal end of the filter to distal end of the filter, ranging between 10 cm and 60 cm, 20 cm and 50 cm, and 30 cm and 40. For example, in some embodiments, filter reservoir 140 has a length of about 35 cm. In some embodiments, filter reservoir 140 has a width or diameter ranging between 10 cm and 60 cm, 20 cm and 50 cm, and 30 cm and 40 cm. For example, in some embodiments housing 102 has a width or diameter of about 35 cm.

Aspects of the present invention relate to materials for an exemplary device 100 and any components thereof. In some embodiments, device 100 comprises any of plastic, metal, composite, glass, ABS, PLA, TPU, PP, PVC, polycarbonate, acrylic, nylon, polyethylene, polytetrafluoroethylene (Teflon), polyethylene terephthalate (Dacron), Buna-N, silicone, polyester, polyurethane, or the like. It should be understood that any material of device 100 may comprise coatings and/or layers as would be known and used by one of ordinary level of skill in the art. This includes, for example, antimicrobial coatings, hydrophobic coatings, UV coatings, anti-static coatings, or the like.

In some embodiments, at least a portion of device 100 is transparent, translucent and/or opaque. For example, in some embodiments, housing 102 comprises a clear material, lid 160 comprises a translucent material, and actuator 126 comprises an opaque material. In some embodiments, at least a portion of device 100 is transparent, including but not limited to, housing 102, lid 160, outer sheath 122 and/or inner sheath 124. In some embodiments, outer sheath 122 and/or inner sheath 124 comprise different colors, delineating markings, and/or graduations to differentiate the first position and second position of valve 120. In some embodiments, device 100 comprises a visual indicator, such as a color-coded marking indicating the various positions of valve 120 and/or sheaths 122 and 124. In some embodiments device 100 further comprises an electronically controlled valve, wherein valve 120 may be actuated and/or toggled electronically.

Aspects of the present invention relate to exemplary methods of use for any disclosed perfusion reservoir device. It should be noted that the present device may utilize methods and/or used in conjunction with other devices known in the art for blood perfusion during procedures such as cardio bypass surgery. In some embodiments, device 100 is used for providing filtered, oxygenated and/or pumped blood to a subject during procedures such as cardiopulmonary bypass surgery. In some embodiments, device 100 may be used to control (i.e. increase or reduce) the volume of blood entering a subject. In some embodiments, device 100 is used with or for an extracorporeal blood circuit. In some embodiments, blood from a subject may enter device 100 through inlet 110, and may pass through either a first or second flow path, and exit the device through outlet 112, and return to the subject. In some embodiments, device 100 may be used as an extracorporeal blood circuit for cardiopulmonary bypass. In some embodiments, when used as an extracorporeal blood circuit for cardiopulmonary bypass, device 100 may be used to rapidly infuse volume in both adult and pediatric populations. In some embodiments, device 100 may be used for thoracoabdominal aneurysm repairs and/or left heart bypass cases, lung transplants, MiECC surgical procedures such as (CABG, Valve, CABG/Valve), and other miscellaneous procedures as healthcare personnel (e.g., a surgeon) sees fit. In some embodiments, device 100 may be used in extracorporeal membrane oxygenation with a level detector attached and other safety devices known in the art as a rapid infuser.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the system and method of the present invention. The following working examples therefore, specifically point out the exemplary embodiments of the present invention, and are not to be construed as limiting in any way the remainder of the disclosure.

### Open-Closed Loop Perfusion Reservoir

During cardiopulmonary bypass, a system is configured to filter, oxygenate, and pump blood to the patient while their heart is being bypassed. There are two types of systems: a closed loop bypass and an open loop bypass. A closed loop system lowers the heparin requirement and reduces the blood/foreign surface interface, but doesn't allow for controlling of patient blood volume. An open loop system allows for control of patient blood volume (via vacuum applied to reservoir) but increases the heparin requirement and the blood/foreign surface interface. Each system has its purpose, but currently they only exist as systems which are meant to be used independently from each other. There are cases when a surgeon wants the ability to swap between an open and closed loop system quickly and efficiently. Currently this is accomplished through a completely custom reconfiguration that requires additional tubing as well as several hemostats which introduce a risk of entraining air posing a serious threat to the patient.

The disclosed device comprises a single venous reservoir system with both an open loop and closed loop mode. In some embodiments, a lever on the inlet would control the switching between these two modes. In some embodiments, when the lever is in the up position, blood would flow directly through the reservoir (see FIGS. 2A through 2C). Blood would enter through the top blue tube. Gravity would make the blood flow downwards through the interface between the blue and green parts and out the bottom of the reservoir. In this position, blood is constrained to this path and cannot enter the greater reservoir space. In some embodiments, when the lever is in the down position, the direct path described above is closed and a path into the greater reservoir is opened (see FIGS. 1A through 1C). Blood again would enter through the top blue tube. Gravity would make the blood flow downwards where it would be blocked by the sealed interface between blue and green parts, flow through the holes in the blue and green parts, forced through the filter, and into the greater reservoir. In some embodiments, the filter is connected and hermetically sealed to the lever toggle at the bottom. It is to be noted that O-rings which seal each position are not shown in the drawings but can be identifiable by the circular notches in their respective parts.

In some embodiments, the disclosed device provides a cardiopulmonary bypass reservoir with both open and closed loop capabilities without the need for reconfiguration. In some embodiments, the disclosed device comprises a valve that may be swapped between an open and closed loop without creating any pooling or stagnation of flow. In some embodiments, the valve passes through the filter interface and directly controls whether fluid passes through filter. In some embodiments, the reservoir shape and valve flow exits allow for a large capacity of total blood volume while requiring only a minimal amount of volume for priming. In some embodiments, the valve comprises on/off positions at three different locations with a single lever toggle (i.e. flow is allowed or restricted at three locations simultaneously with the toggling of the lever)

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A blood perfusion reservoir device, comprising:
a housing forming proximal and distal chambers, the housing having proximal and distal openings;
a valve having a proximal end, a distal end and a length forming a lumen therebetween, the valve being positioned within the housing such that the proximal end passes through the proximal opening of the housing, the distal end passes through the distal opening of the housing, and the length passes through the proximal and distal chambers of the housing, wherein the valve is configurable between a first and second position, the first position forming a first flow path and the second position forming a second flow path;
a filter reservoir comprising a frame with a filter media forming proximal and distal filter regions with a proximal opening, and positioned within the housing fixedly attached to the valve and surrounding a length of the valve;
wherein the first flow path fluidly connects the proximal and distal openings, and the flow bypasses the proximal and distal chambers, the proximal and distal filter regions, and the second flow path fluidly connects the proximal and distal openings, and the flow passes through proximal and distal chambers and the proximal and distal filter regions.

2. The device of claim 1, wherein the valve comprises at least one outer sheath and at least one inner sheath, with the inner sheath inserted into the outer sheath and slidably movable relative to the outer sheath and forming the lumen therethrough, each sheath comprising one or more distal openings, and one or more lateral openings along the length of each sheath.

3. The device of claim 2, wherein the sheaths when configured to the first position of the valve allow the first flow path enabling fluid communication between the proximal and distal openings of the housing through the one or more distal openings of the sheaths.

4. The device of claim 3, wherein the sheaths when configured to the second position of the valve allow the second flow path enabling fluid communication between the proximal and distal openings of the housing through the one or more lateral openings of the sheaths.

5. The device of claim 4, wherein the first flow path is defined as a fluidly connected path entering the proximal opening of the housing, proceeding through the lumen formed by the inner sheath, exiting the distal opening of the inner sheath and entering the outer sheath, proceeding through the lumen formed by the outer sheath and exiting the distal opening of the outer sheath, and out the distal opening of the housing.

6. The device of claim 5, wherein the second flow path is defined as a fluidly connected path entering the proximal opening of the housing, proceeding through the lumen formed by the inner sheath, exiting the lumen of the inner sheath through the one or more lateral openings, passing through the one or more lateral openings of the outer sheath into the distal filter region filling the proximal filter region as necessary, through the filter media, into the distal chamber and filling the proximal chamber as necessary, and out the distal opening of the housing.

7. The device of claim 2, wherein the outer sheath slidably moves axially between the first and second positions of the valve.

8. The device of claim 2, wherein the housing and filter reservoir comprise proximal openings configured to receive one or more lids covering the proximal openings.

9. The device of claim 8, wherein the proximal openings of the inner and outer sheaths extend proximally through the one or more lids.

10. The device of claim 1, further comprising at least one opening in the housing configured to apply pressure or vacuum to the first and second interior volumes.

11. The device of claim 2, further comprising a proximal button fixedly attached to a position at the proximal end of the outer sheath, configured to lift or depress the outer sheath to configure the valve between the first and second positions, respectively.

12. The device of claim 11, wherein the valve further comprises a spring positioned between the button and the housing, configured to apply a preload to the button.

13. The device of claim 1, wherein the housing is a shape selected from the group consisting of: frustoconical, cone, cylinder, sphere, and hexahedron.

14. The device of claim 1, wherein the filter reservoir is a shape selected from the group consisting of: frustoconical, cone, cylinder, sphere, and hexahedron.

15. The device of claim 1, wherein the housing comprises a material selected from the group consisting of: plastic, metal, composite, glass, ABS, PLA, TPU, PP, PVC, polycarbonate, acrylic, nylon, polyethylene, polytetrafluoroethylene (Teflon), and polyethylene terephthalate (Dacron).

16. The device of claim 1, wherein the filter reservoir comprises a material selected from the group consisting of: plastic, metal, composite, glass, ABS, PLA, TPU, PP, PVC, polycarbonate, acrylic, nylon, polyethylene, polytetrafluoroethylene (Teflon), and polyethylene terephthalate (Dacron).

17. The device of claim 1, wherein the housing has a proximal to distal length ranging between 20 and 50 cm, and a width or diameter ranging between 10 and 40 cm.

18. The device of claim 1, wherein the filter reservoir has a proximal to distal length ranging between 10 and 40 cm, and a width or diameter ranging between 5 and 30 cm.
